(19) United States
(12) Patent Application Publication
(10) Pub. No.: US 2023/0355176 A1
(43) Pub. Date: Nov. 9, 2023

(54) ELECTRICALLY CONDUCTIVE MATERIALS

(71) Applicant: Battelle Memorial Institute, Columbus, OH (US)

(72) Inventors: Matthew Colachis, Columbus, OH (US); Katherine M. Palmer, Galloway, OH (US); Samuel Colachis, Columbus, OH (US); Amy M. Heintz, Pacific Grove, CA (US); Charli Ann Hooper, Pittsburgh, PA (US); Collin Dunlap, Columbus, OH (US); Nicholas Annetta, Columbus, OH (US)

(21) Appl. No.: 17/946,115

(22) Filed: Sep. 16, 2022

Related U.S. Application Data
(60) Provisional application No. 63/249,562, filed on Sep. 28, 2021.

(51) Int. Cl. A61B 5/00; B32B 5/18; B32B 7/12; B32B 5/24; B32B 5/26; B32B 5/02

(52) U.S. Cl. CPC A61B 5/6804; B32B 5/18; B32B 7/12; B32B 5/245; B32B 5/26; B32B 5/024; B32B 5/022; A61B 2503/40; A61B 2562/125; B32B 2260/021; B32B 2260/04; B32B 2307/516; B32B 2266/06; B32B 2437/00; B32B 2535/00; B32B 2307/202; B32B 2264/108; B32B 2266/0278

(57) ABSTRACT

The present disclosure relates to a variety of devices, systems, and methods of utilizing mixed-ionic-electronic conductor (MIEC) materials which are adapted to function with an applied current or potential. The materials, as part of a circuit, can placed in contact with a part of a human or nonhuman animal body.

### CROSS-REFERENCE TO RELATED APPLICATIONS

This application claims priority to U.S. Provisional Patent Application Ser. No. 63/249,562, filed on Sep. 28, 2021, which is incorporated by reference in its entirety.

### BACKGROUND

The present disclosure relates to electrically conductive materials that can be useful in wearable electronic garments. Such garments can be used for applications such as electrophysiology measurement, electromyography (EMG) measurement, transcutaneous electrical nerve stimulation (TENS), neuromuscular electrical stimulation (NMES), and functional electrical stimulation (FES), sports rehabilitation, and other similar applications.

Wearable electronic garments usually include electrodes. An electrolytic conductive gel disposed on the electrode bridges the ionic collector (skin) with the current collector to reduce the potential drop between the electrode and the skin of the user. If no conductive gel is used, or if the conductive gel is insufficient or gets wiped off (for example during donning of the garment), then the user may experience pain or skin irritation, for example due to electrical arcing between the metal electrode and the skin. Conductive gels can also introduce other problems such as allergic reactions, discomfort, and variable signal quality that changes as a function of gel loading. The conductive gel can also be messy and difficult to wash away. The cost of hydrogels, supply maintenance, setup time, and ease-of-use of these gels are additional problems.

Certain improvements are disclosed herein.

### BRIEF DESCRIPTION

The present disclosure relates to composite materials which include a “Mixed Ionic-Electronic Conductor” or (MIEC) material or layer. The MIEC material is electrically conductive, and is incorporated into a textile or foam substrate to form an electrically conductive composite material. The composite material is also flexible and conformal, is comfortable, can make good contact with the skin, is easy to don and remove. The composite material is soft and can also be cast into any desired shape, including a sleeve or a custom fit. This permits use of the MIEC material to be used in many different industries and applications.

The present disclosure also relates to devices or systems incorporating such composite materials. The present disclosure also relates to methods for treating mammals such as humans or nonhuman animals using the devices or systems, especially in combination with particular materials and conditions.

These and other non-limiting aspects of the present disclosure are more particularly described below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is an illustration of a sheet made of MIEC material.

FIG. 2 is another illustration of a sheet made of MIEC material and having a lip.

FIGS. 3A and 3B show an illustration of two successive steps in making a molded MIEC sheet according to an embodiment disclosed herein.

FIG. 4 is an illustration of a composite material having a MIEC material layer and a textile or foam substrate.

FIG. 5A is an exterior perspective view of a mold for producing a MIEC material having a tubular shape. FIG. 5B is a cross-sectional view of the mold.

FIG. 6 is a plan view of an inner surface of a portion of an electronic stimulation garment with a thin sheet of MIEC material applied thereon.

FIG. 7 is an isolation view of a compression sleeve formed from MIEC material.

FIG. 8 is a view showing the compression sleeve of FIG. 7 worn on an arm.

FIG. 9 is a view showing the compression sleeve of FIG. 8 along with an electronic stimulation garment disposed over the compression sleeve, with a portion pulled back to show the inner surface thereof having electrodes disposed on the inner surface.

### DETAILED DESCRIPTION

A more complete understanding of the devices and methods disclosed herein can be obtained by reference to the accompanying drawings. These figures are merely schematic representations based on convenience and the ease of demonstrating the existing art and/or the present development, and are, therefore, not intended to indicate relative size and dimensions of the assemblies or components thereof.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function. In the following specification and the claims which follow, reference will be made to a number of terms which shall be defined to have the following meanings.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named components/steps and permit the presence of other components/steps. However, such description should be construed as also describing devices or methods as “consisting of” and “consisting essentially of” the enumerated components/steps, which allows the presence of only the named components/steps, and excludes other components/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value. All ranges disclosed herein are inclusive of the recited endpoint.

The term “about” can be used to include any numerical value that can vary without changing the basic function of that value. When used with a range, “about” also discloses the range defined by the absolute values of the two endpoints, e.g. “about 2 to about 4” also discloses the range “from 2 to 4.” The term “about” may refer to plus or minus 10% of the indicated number.

Some terms used herein are relative terms. For example, the terms “top” and “bottom” are relative to each other. These relative terms are intended to encompass different orientations of the device in use or operation, and should be interpreted accordingly. The terms “horizontal” and “vertical” are used to indicate direction relative to an absolute reference, i.e. ground level. However, these terms should not be construed to require structures to be absolutely parallel or absolutely perpendicular to each other.

The present disclosure relates to composite materials which include a “Mixed Ionic-Electronic Conductor” or (MIEC) material or layer. The MIEC material is electrically conductive, and is incorporated into a textile or foam substrate to form an electrically conductive composite material. The resulting composite material can be used in many different applications.

Components and Methods of Making

The “mixed ionic-electronic conductor” or (MIEC) material or layer is formed from an interconnected network of electrical conductors and ionic conductors in an elastomeric matrix. The electrical conductors and the ionic conductors are embedded in the matrix in such a way that the electrical and ionic elements achieve percolation, i.e., a continuous interconnected network, at lower loading than would be achieved by simple random mixing. This allows superior electrical performance to be achieved while retaining good mechanical properties. The resulting material or layer provides: (1) high surface area for efficient capacitive charge-discharge; (2) high ionic conductivity for low interfacial charge transfer, where the interfacial charge transfer can be viewed as made up of resistive and capacitive response; (3) low ohmic resistance; and (4) excellent flexibility and toughness.

The MIEC material comprises an elastomeric phase which is formed from polymer particles. Examples of suitable materials for the elastomeric phase include elastomeric polymers such as acrylonitrile butadiene rubber (NBR), natural rubber, silicone, silicone acrylic, or polyurethane. Other suitable polymers include polyvinylidene fluoride or polyvinylidene chloride or other fluoropolymers.

The MIEC material also comprises an electrical conductor and an ionic conductor. Suitable electrical conductors are those that have high aspect ratio and are readily dispersed into aqueous solutions and include carbon nanotubes, graphene and graphite structures, and metal nanowires, and combinations thereof. In particular embodiments, the electrical conductors have a number average aspect ratio of height to the smallest width dimension of at least 10.

The term “carbon nanotube” or “CNT” includes single, double, and multiwall carbon nanotubes and, unless further specified, also includes bundles and other morphologies. The CNTs can be any combination of these materials, for example, a CNT composition may include a mixture of single and multiwall CNTs, or it may consist essentially of DWNT and/or MWNT, or it may consist essentially of SWNT, etc. CNTs have an aspect ratio (length to diameter) of at least 50, preferably at least 100, and typically more than 1000. The CNTs may be made by methods known in the art such as arc discharge, CVD, laser ablation, or HiPco.

Suitable ionic conductors include glycosaminoglycans such as sodium hyaluronate (a salt of hyaluronic acid), fluorosulfonic acids like Nafion™, sulfated polysaccharides and other mucoadhesive type compounds, or other phosphonic polyvinylsulfonic acids. Likewise, anisotropic ionic conductive particles like graphene oxide and modified graphene oxide may be used. Combinations thereof are also contemplated. In some embodiments, hyaluronic acid (HA) is preferred due to its tendency to hydrate with the skin, improving the skin contact with the MIEC material.

Glycosaminoglycans are long unbranched polysaccharides consisting of a repeating disaccharide unit. The repeating unit (except for keratan) consists of an amino sugar (N-acetylglucosamine or N-acetylgalactosamine) along with a uronic sugar (glucuronic acid or iduronic acid) or galactose. Glycosaminoglycans are highly polar. Anionic glycosaminoglycans are characterized by having at some hydroxyl protons replaced by a counter ion; typically an alkali or alkaline earth element. Examples of glycosaminoglycans include: β-D-glucuronic acid, 2-O-sulfo-β-D-glucuronic acid, α-L-iduronic acid, 2-O-sulfo-α-L-iduronic acid, β-D-galactose, β-O-sulfo-β-D-galactose, β-D-N-acetylgalactosamine, β-D-N-acetylgalactosamine-4-O-sulfate, β-D-N-acetylgalactosamine-6-O-sulfate, β-D-N-acetylgalactosamine-4-O, 6-O-sulfate, α-D-N-acetylglucosamine, α-D-N-sulfoglucosamine, and α-D-N-sulfoglucosamine-6-O-sulfate. Hyaluronan is a particularly preferred glycosaminoglycan and representative of its class.

In preferred embodiments, carbon nanotubes are the electrical conductors and hyaluronic acid (HA), or other glycosaminoglycans, along with residual atmospheric moisture and ions, is the ionic conductor.

In some embodiments, the MIEC material comprises 0.1 to 2 wt % CNTs, including 0.2 to 1 wt %, and in some embodiments 0.5 to 0.8 wt % CNTs (by weight of the as dried MIEC material). In some embodiments, the MIEC material comprises 0.1 to 5 wt % glycosaminoglycan, preferably 0.4 to 4 wt %, and in some embodiments 0.7 to 3 wt % glycosaminoglycan. In some embodiments, the mass ratio of glycosaminoglycan to CNT in the dried MIEC material is in the range of 0.5 to 10, preferably 4 to 8, and in some embodiments 1.5 to 2.5. In some embodiments, the MIEC material comprises at least 0.01 wt % Na, or 0.01 to 2 wt % Na, and in some embodiments 0.1 to 1 wt % Na. This may occur, for example, when the ionic conductor is sodium hyaluronate. It is further contemplated that the MIEC material can be characterized by any one or any combination of these properties. The remainder of the MIEC material is formed from the elastomeric phase.

The MIEC material may be formed from a solution, dispersion, or slurry of the polymer particles, electrical conductor, ionic conductor, and water. Other additives may also be present in the solution/dispersion/slurry that is used to form the MIEC material, and may subsequently end up in products formed therefrom. For example, dispersants such as carboxymethylcellulose (CMC) may be used to better disperse the electrical and ionic conductors. Examples of other additives may include colorants (e.g. dyes or pigments), fillers, stabilizers, flame retardants, plasticizer, mold release agent, and antioxidants.

In some particular embodiments, a small amount of adhesive, such as Pros-Aide®, may be added to the MIEC dispersion to allow for stronger adhesion of the MIEC material to either the subject's skin or an electronic stimulation garment. Loadings of 0.01 wt % to 2 wt % may allow for this desired adhesion. Pros-Aide® contains an acrylic emulsion which acts as an adhesive, along with water, glycerin, guar gum, sorbitol, and benzyl alcohol.

The morphology of the interconnected network in the MIEC material may be controlled by using a polymer latex, also called an emulsion, in which the polymer particles are dispersed in an aqueous phase, to template the organization of the electrical and ionic conductors. In such a dispersion, at least 90 mass % of the polymer particles are preferably in the range of 50 nm to 10 μm in diameter.

The electrical and ionic conductors are added to the latex so that they are dispersed in the aqueous phase. Methods known in the art for balancing the pH and selecting any necessary dispersing agents can be used. By adding electrical and ionic conductors to the dispersed phase of the latex, the conductors tend to coat the surface of the polymer particles, but not penetrate. As the latex is dried, the conductors tend to be confined at the interfaces, creating an interconnecting network, where the major elastomeric phase is formed from the polymer particles and a connected thin layer phase is formed from the electronic conductors and the ionic conductors.

The dispersion is cast and the volatiles (e.g., water) allowed to evaporate. During evaporation, the polymer particles coalesce to form a continuous fill. This process is commonly used, for example for creating nitrile gloves.

The morphology of this network can be modified by changing the particle size of the polymer particles in the latex. Larger polymer particle sizes require less conductor to produce an interconnected phase. The film formation temperature is also a tunable parameter that can used to modify the kinetics to achieve various kinetically trapped states. Other methods to achieve better than random mixing include self-assembling or self-stratifying coatings.

Preferably, the MIEC material has high conductivity of at least 1000 mS/cm, preferably at least 2000 mS/cm, or in the range of 2000 mS/cm to about 4000 mS/cm is desirable.

In some preferred embodiments, the MIEC material has high moisture retention, such that the composite material may absorb at least 20% by mass water (wherein 100% is equivalent to the weight of the dry composite), including up to 50% by mass water, or in some further embodiments 20% to 50%, or 35% to 50% water.

The MIEC material may be cast into a layer or sheet by itself. This may be, for example, by pouring the polymeric dispersion (including polymer particles, electrical conductor, ionic conductor, and water) into a mold and subsequently curing to obtain a sheet of the MIEC material.

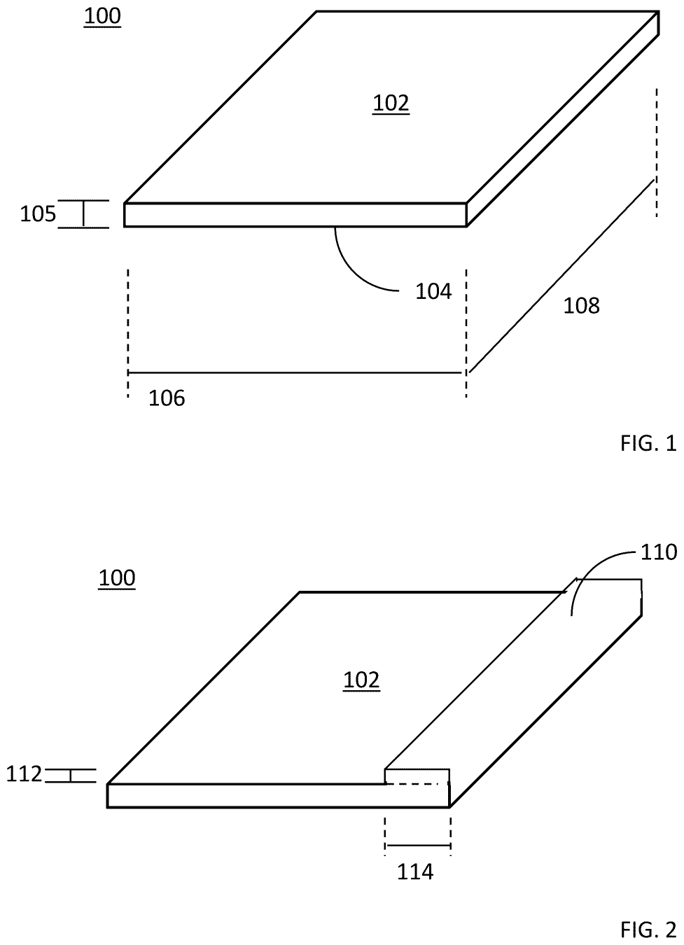

FIG. 1 is an illustration of such a sheet 100. The sheet has two major surfaces 102, 104 on opposite sides of the sheet. The thickness 105 of the sheet is measured between the two major surfaces, and is generally contemplated to be constant throughout the sheet. The sheet has a length 106 and a width 108. In particular embodiments, the thickness of the sheet may be at least 0.1 millimeters, or at least 0.2 mm, or may range from 0.1 mm to 2 mm, or from 0.1 mm to 1 mm. In particular embodiments, the length and width are independently at least 5 centimeters (cm), including at least 10 cm, and may range up to 50 cm.

FIG. 2 is another illustration of a molded MIEC material. To obtain this shape, the mold may include borders, such that the final shaped sheet 100 has a lip 110. After the MIEC material is cured and released from the mold, the “lip” can be folded over the sheet, resulting in a border on the edge of the final sheet which allows for increased anchorage to the electronic stimulation garment (not shown). In FIG. 2, only one lip is present, and the lip is shown folded over. Of course, lips may be present on two or more sides of the sheet as well. In this folded-over position, the lip has a height 112 and a depth 114. The height is measured perpendicular to the major surface 102 of the sheet, and the depth is measured in the direction from the edge of the sheet into the center of the sheet. In particular embodiments, the height of the lip may range for example from 1 mm to 5 mm. In specific embodiments, the depth of the lip is at least 2 mm, or at least 5 mm, or from 2 mm to 10 mm). The molded MIEC material is electrically conductive. The lip may be useful, for example, for increasing anchorage to another surface, such as a textile or foam substrate or to another electronic stimulation garment.

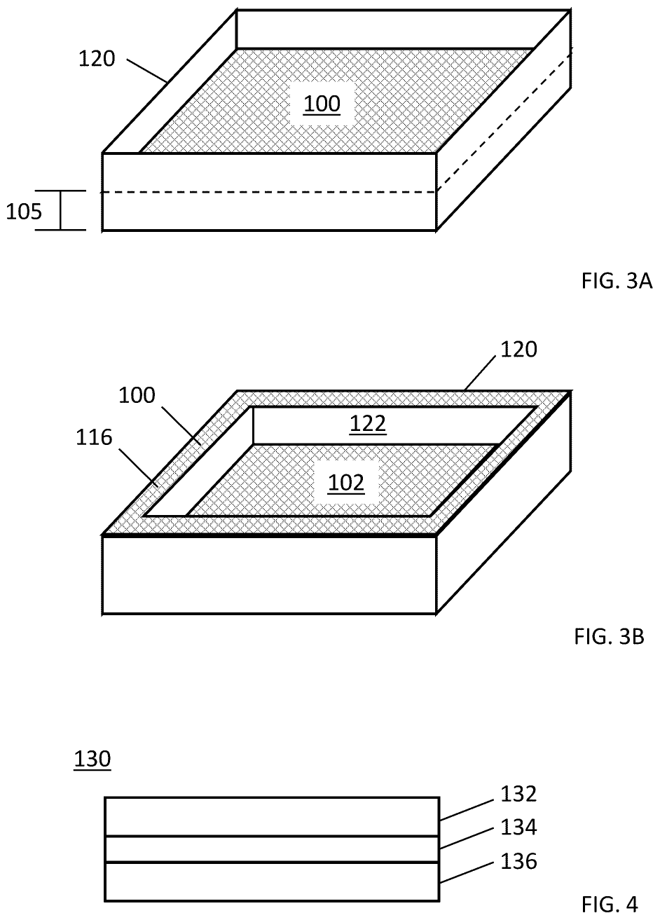

FIG. 3A and FIG. 3B show another illustration of a molded MIEC material. In this illustration, the molded MIEC sheet is made using two separate steps. In the first step as seen in FIG. 3A, a sheet 100 with a uniform thickness is made and cured in an external mold 120. It is noted that the external mold is not completely filled, and is taller than the sheet 100. The thickness 105 of the sheet relative to the external mold is shown in dotted line. In the second step as seen in FIG. 3B, an internal mold 122 is placed upon the sheet 100 and within the external mold 120. Additional MIEC solution/dispersion/slurry is then poured between the internal mold and the external mold and cured. This results in a MIEC sheet 100 that has a border 116 around the perimeter of the major surface 102, which may be thicker and more durable than the lip described in FIG. 2.

The sheet can also be folded or otherwise shaped into any desired shape, such as a sleeve which can be used in conjunction with an electronic stimulation garment. In some embodiments, the electrical conductors in the MIEC material can be aligned in a particular direction, to create anisotropic conductivity. For example, carbon nanotubes (CNTs) can be aligned by application of a strong electrical or magnetic field during the curing of the dispersion. Other methods for aligning carbon nanotubes are known and may be applied as well. in this regard, randomly oriented CNTs permit transverse current spreading across the conductor in EMG and FES applications. In EMG applications, current spreading distributes the muscle activity across many electrodes of the electronic stimulation garment and may harm decoding accuracy due to reduce specificity and transverse signal loss. In FES applications, current spreading can reduce the stimulating pattern specificity by delivering current to off-target muscles. Alignment would be useful in mitigating these issues by promoting signal transfer through the MIEC material and resisting conduction laterally across the interface. Only partial alignment may be desired to minimize the loss in effective void space, or the nanotube volume fraction may be increased to compensate for void space changes.

In some particular embodiments, at least 30 wt % of the CNTs are disposed on a major surface or within the 10% of the thickness near a major surface.

In other embodiments, the MIEC material is stretched by extending the molded MIEC material and allowing the material to contract. Desirably, the original unstretched MIEC material possesses a conductivity of 1000 mS/cm to about 3000 mS/cm, and the conductivity changes by less than 10% after 5 strain cycles of stretching the material by 50% of its length and allowing the material to contract.

In other specific embodiments, the MIEC material possesses a ratio of partial conductivity of a charge carrier to the total conductivity, transference number, tᵢ of at least 0.10, preferably at least 0.13, and in some embodiments in the range of 0.10 to about 0.20 or from 0.15 to about 0.20.

In some embodiments, the sheet of MIEC material is made from multiple layers which are individually fabricated. The sheet has a top and bottom surface, with each layer having an increasing weight ratio of ionic conductor to electrical conductor from the top surface to the bottom surface. This layered sheet may have at least 3 layers, or at least 5 layers. The conductivity of the resulting layered sheet thus has a gradient within the sheet.

In some embodiments, the CNT network layer formed in the MIEC material may be continuous over the entire sheet. In some other embodiments, the CNT network layer is formed of rows of CNT networks separated by rows of polymer (such as CNTs deposited in a grooved polymer substrate).

The MIEC material or layer can also be combined with a textile or foam substrate to obtain an electrically conductive composite material. Such substrates may be made from materials that include cheesecloth; cotton; polymers such as Spandex® (i.e. a polyether-polyurea copolymer), acrylonitrile-butadiene rubber (NBR), latex rubber or natural rubber, polyurethane (low-density or high-density), polyvinylidene fluoride, polyvinylidene chloride, ethylene-vinyl acetate (EVA), polyethylene, polychloroprene, polyimide, polypropylene, polystyrene, polyvinyl chloride (PVC), silicone or silicone acrylic; or woven or non-woven fabrics. The substrate can be formed from threads, fibers, yarn, etc. made of such materials. Foam substrates can also be made from similar polymeric materials, or biological materials such as soy oil, cellulose, polylactic acid (PLA), or polyglycolic acid (PGA). The foam substrate is typically an open-cell foam to permit penetration and absorption of the MIEC material. In some alternative embodiments, the substrate may be a yarn with a high length/diameter ratio, which might be useful for producing unique flexible textile sensors and circuits.

In some embodiments, a molded MIEC material layer or sheet is directly adhered to the textile or foam substrate. This may be done, for example, using an adhesive. An illustration of the resulting electrically conductive composite material 130 is shown as a cross-section in FIG. 4. Here, the MIEC material 132 is adhered to a foam substrate 136 by adhesive layer 134. In this regard, the term “directly” means there are no other layers (besides the adhesive) between the MIEC material layer and the substrate. The adhesive itself is also electrically conductive, for example an epoxy. In another alternative embodiment, the MIEC solution/dispersion/slurry is applied in an uncured form to one side of the substrate and then cured.

In other embodiments, the textile or foam substrate is impregnated with the MIEC material. This can be done, for example, by soaking the textile or foam substrate in an aqueous solution or dispersion or slurry containing the polymer particles, electrical conductor, and ionic conductor. The textile or foam substrate can be compressed or stretched, and then released multiple times during the soaking to encourage impregnation. The soaked substrate is then cured to obtain the electrically conductive composite material. It is also contemplated the textile or foam substrate could be vacuum sealed together with the MIEC solution/dispersion/slurry, resulting in the MIEC being impregnated into the textile or foam substrate. The interconnected network of electrical conductors and ionic conductors penetrates throughout the textile or foam substrate.

Beyond a sheet form factor, it may be desirable to make another form factor from the MIEC solution/dispersion/slurry prior to curing to allow for a closed system, such as in the shape of an arm band sleeve or an amputee sleeve. Such form factors would allow for total coverage of the limb and the electrodes of the electronic stimulation garment, easy don and doff of the MIEC material, and eliminate the need to anchor the MIEC material to the electronic stimulation garment.

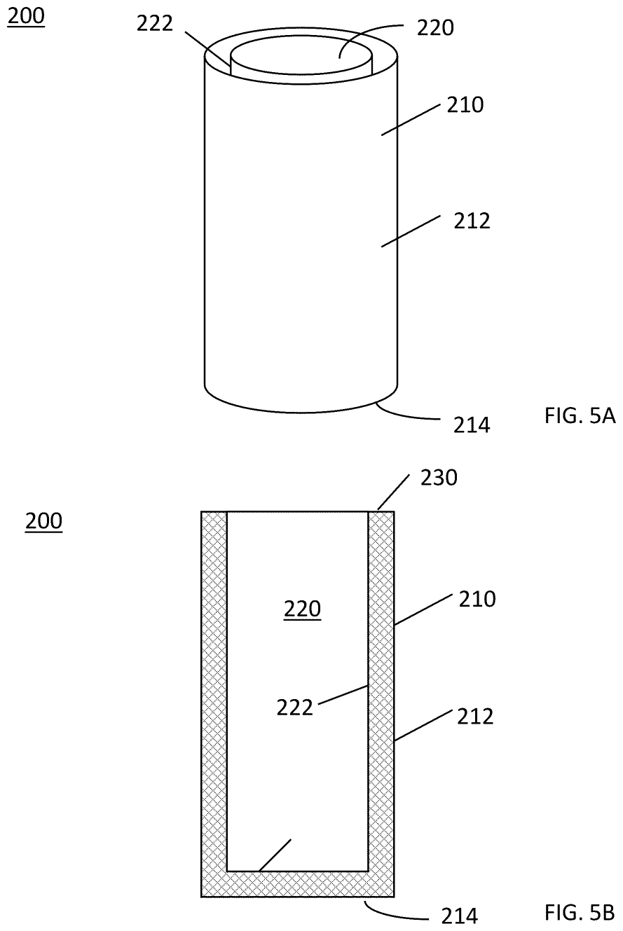

FIG. 5A and FIG. 5B are an illustrative example of a mold 200 that could be used to produce a cured MIEC material with a tubular shape. FIG. 5A is an exterior perspective view, and FIG. 5B is a cross-sectional view. The mold 200 includes an external half 210 (also called a cavity or a stationary side) and an internal half 220 (also called a core or a moving half). The external half 210 includes a sidewall 212 and a bottom wall 214. The internal half 220 is solid and also includes a sidewall 222 and a bottom wall 224. As best seen in FIG. 5B, the bottom wall 224 of the internal half does not contact the bottom wall 214 of the external half. The MIEC material 230 is also shown here, between the two halves of the mold. In some embodiments, one or both halves of the mold can be heated, to speed up the curing process. As a result, the shape of the cured MIEC material can be described as a tube that is closed at one end and open at the opposite end. A similar mold could be used to make a bowl-shaped cured MIEC material as well. After removal from the mold, holes could be cut into the cured MIEC material as desired, for example to allow for the fingers and the thumb.

The MIEC material could also be cast molded. In this molding process, the MIEC solution/dispersion/slurry is poured into a hot mold and then moved to an oven to cure. The mold is only removed when the material has completely hardened. Most cast molded rubber must be cured twice.

An alternative method for making a bowl-shaped cured MIEC material would be to put the MIEC solution/dispersion/slurry into a bowl and rotate the bowl during the curing process. As the bowl rotates, the slurry would travel up the sides of the bowl. The thickness of the resulting cured MIEC bowl might vary from the bottom of the bowl to the top of the bowl.

Other form factors could be made from an oversized cured two-dimensional MIEC sheet or composite. The ends/edges of the MIEC sheet could be joined to form the desired shape, for example through joining methods such as, but not limited to, sewing, chemical reaction, or heating. The resulting form factor could be a tubular shape or another closed geometric shape. Alternatively, additional MIEC solution/dispersion/slurry could be applied to a cured or almost-cured MIEC sheet or composite, which would cause the ends/edges to stick to each other. The ends/edges of the MIEC sheet could also be joined with an adhesive, such as a cyanoacrylate.

As another method, the MIEC material could be vulcanized, to make it more durable for molding and/or stronger to allow for sewing the ends together. This may also result in less tearing of the MIEC sheets too. This could be done by pressure vulcanization, which involves heating the MIEC material with sulfur under pressure at an elevated temperature of, for example, 150° C. This could also be done by free vulcanization, which simply involves passing very hot steam or air through the cured MIEC sheet. Vulcanizing the MIEC material may result in the materials achieving different mechanical and electrical properties.

It should be noted that depending on the techniques used to join the ends/edges of the MIEC sheet, the electrical and mechanical properties of the homogenous portion of the MIEC sheet may differ from the joint, especially where mechanical manipulation or heating is involved.

Uses and Applications

The MIEC material, either as a sheet or other shaped structure, or as part of the electrically conductive composite material with the textile or foam substrate, can be used as a bridge between the skin of the user and the electrodes of an electronic stimulation garment. This reduces the likelihood of electrical arcing during electrical stimulation. This also decreases the potential for “pain” when using the electrodes and decreases biting (electrical discharge through the backside of the garment at the location of the electrode) when used in an electrical stimulation application.

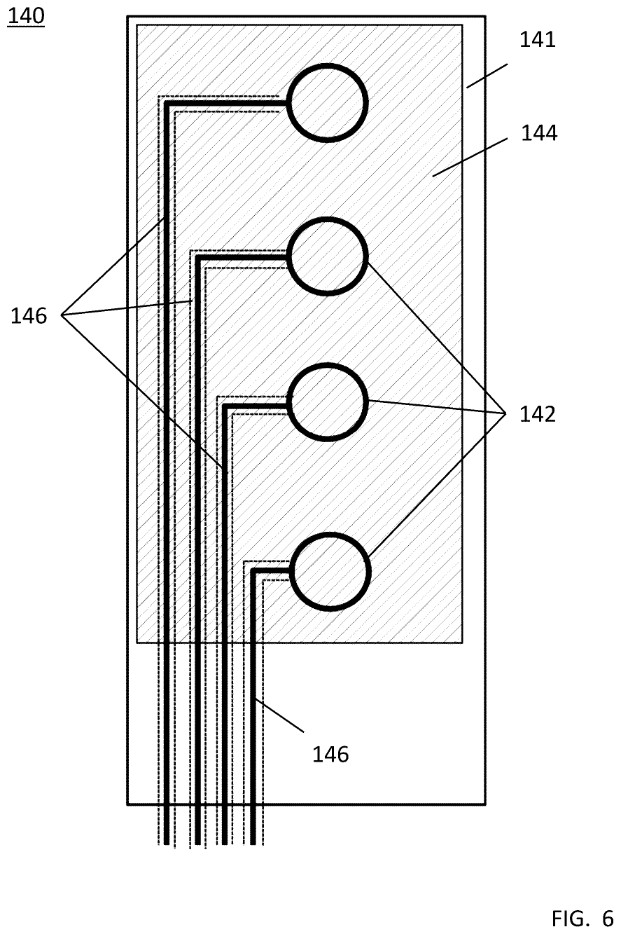

FIG. 6 is an illustration where a thin sheet 144 including MIEC material is used in conjunction with a first textile layer 141 that has a plurality of electrodes 142 attached thereto. Wiring 146 leading to each electrode is also shown. The first textile layer and electrodes may be part of an electronic stimulation garment 140, which may have the form factor for example of a sleeve. As seen here, the sheet 144 extends over multiple (possibly all) electrodes 142 of the electronic garment. As long as the sheet is sufficiently thin, electrical conduction between neighboring electrodes 142 through the thin sheet 144 is avoided. In one approach, the thin sheet 144 can be secured to the first textile layer 141 of the electronic garment. For example, the thin sheet 144 can comprise a layer of MIEC material. In other embodiments, the thin sheet 144 can comprise a textile or foam substrate which has been impregnated with MIEC material, which is then secured to the first textile layer 141, for example by sewing or by use of adhesive. This latter approach effectively forms a two-ply garment, including the electrically nonconductive first textile layer 141 as one ply and the thin sheet 144 as the second ply, with the electrodes 142 disposed therebetween.

The thin sheet 144 comprising the cured MIEC material can be anchored to the first textile layer 141 of the electronic garment using multiple different methods. For example, an adhesive such as a cyanoacrylate, could be used to bond the thin sheet to the first textile layer. The thin sheet could be sewn to the first textile layer, which would permanently join them together. A hook-and-loop fastener, such as Velcro®, could be used to join the thin sheet to the first textile layer. This would provide a removable option for changing the thin sheet.

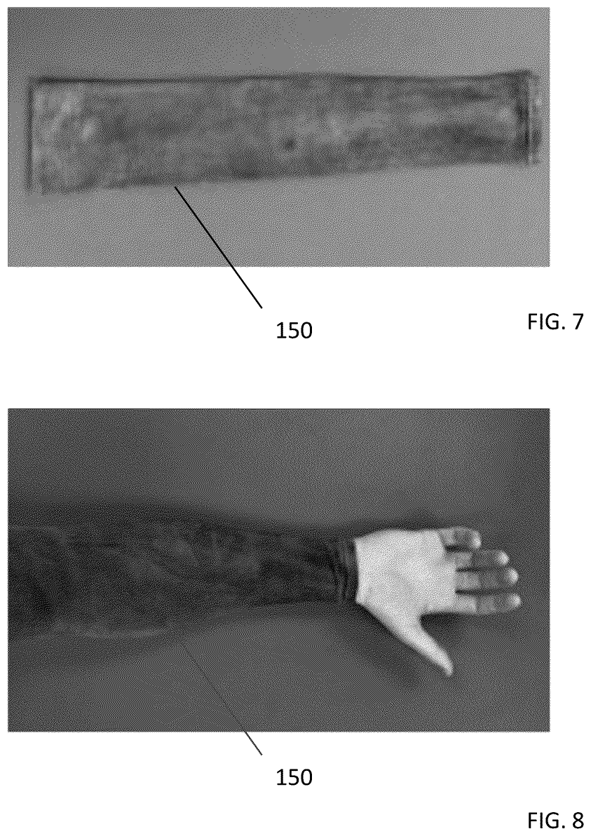

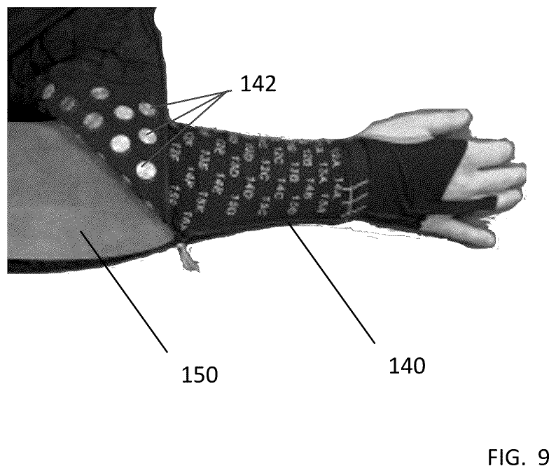

As illustrated in FIGS. 7-9, in other embodiments the thin sheet is embodied as a separate compression sleeve 150 or the like, which is disposed between the skin and the electronic stimulation garment. FIG. 7 shows the compression sleeve 150 in isolation. The compression sleeve 150 is a garment that when worn on target anatomy compresses against that target anatomy of the body, and is coated and/or impregnated with the MIEC material. This is shown in FIG. 8, where the compression sleeve 150 is worn on an arm. The compression sleeve is elastic and does not rip easily. In one nonlimiting illustrative embodiment, the compression sleeve comprises from 88 wt % to 92 wt % nylon/polyamide/polyester fibers and 8 wt % to 12 wt % Spandex®. FIG. 9 shows the compression sleeve 150, with the electronic stimulation garment 140 also in the form of a sleeve disposed over the compression sleeve 150, with a portion of the electronic garment 140 pulled back to reveal its inner surface with the electrodes 142.

In some embodiments, an adhesive (such as Pros-Aide) can be used to anchor the MIEC material sheet to the compression sleeve. The use of adhesive allows the MIEC material sheet to be attached and released from the compression sleeve easily and quickly. This allows a user to change the MIEC material sheet quickly, in contrast to prior methods which required time-consuming application of conductive gel to the user's body. The MIEC material provides good electrical contact with the skin even in the absence of a conductive gel. In some embodiments, the contact with the skin can be improved using a conductive spray, such as Signa Spray®.

More generally, an electronic stimulation garment generally includes a textile layer and electrodes. The textile layer may be a woven fabric or the like, which in some embodiments is an elastic fabric such as Spandex™, Lycra ™, elastane or so forth comprising synthetic fibers with high elasticity making the electronic stimulation garment as a whole elastic. The garment could have the form factor of an armband, wristband, leg band, a sleeve covering part or all of the arm and/or wrist and/or hand, a sleeve covering part or all of a leg and/or ankle and/or foot, a vest covering the torso, a skullcap, various combinations thereof, and/or so forth. In such various types of electronic garments, the mechanism for securing or fastening the garment to the anatomy of the wearer may vary, e.g. an electronic garment in the form of a sleeve can be an elastic sleeve in which fabric of the sleeve is elastic and the sleeve is held on the arm or leg or other anatomy by the elastic sleeve compressing against the arm. Some further examples of suitable form factors of wearable electronic garments are described in U.S. Pat. Nos. 9,884,178 and 9,884,179, both of which are incorporated herein by reference in their entireties. Again, these are merely nonlimiting illustrative examples. The electronic stimulation garment is typically wired to a pigtail or bundler or other cable that connects with electronics such as an electrical stimulator or readout electronics. In some designs, a portion of the electronics may be integrated with the garment itself, for example as electronic cards or modules embedded therein or attached thereto.

The electrically conductive MIEC material, either as a sheet by itself, or as part of a composite material with a textile or foam substrate, has a wide use of applications such as in sports rehabilitation, relaxation therapy, sports therapy, electrocardiogram (ECG) measurements, fetal scalp electrodes, electroencephalography (EEG) electrodes, functional electrical stimulation (FES), neuromuscular electrical stimulation (NMES), transcutaneous electrical nerve stimulation (TENS), haptic operations, electromyography (EMG), more generally electrophysiology measurements (e.g., ECG, EEG, EMG, et cetera), in a pacemaker or an automated external defibrillator (AED), and so forth.

One phenomenon noticed during stimulation was that when the MIEC material was compressed, the FES intensified. There may be a linear relationship with the compressive distance of the MIEC material on the intensity of the current, allowing for higher muscle movement for FES at lower currents.

In some embodiments, spraying Signa Spray (a saline solution) onto the soft MIEC sheet or a person's skin eliminates any type of biting and performs almost identical to a hydrogel sheet with much fewer side effects and increased comfort.

The present disclosure also relates to methods of treating a mammal such as a human or nonhuman animal; methods of administering a treatment; methods of recording or stimulating a nerve in a mammal such as a human or a nonhuman animal; and methods of obtaining an EEG, ECG, or EMG. Generally, a composite material including the MIEC material is applied to the mammal, for example to a limb. One or more electrodes which have been applied to the MIEC are then activated to provide stimulation. Alternatively, the electrodes are used for measurement.

Any peripheral nerve that can be reliably activated through the skin and affect physiological function may be used with the MIEC material described herein. For example, the MIEC material can be used with peripheral nerve stimulation-based therapies to treat disease (e.g. auricular nerve stimulation for atrial fibrillation or trigeminal nerve stimulation for migraine). The nerves that can be specially targeted for non-invasive nerve stimulation are many, and some are listed above for example cases.

Advantages

One useful attribute of the MIEC material and the composite materials disclosed herein are their application to various wearable and comfortable forms, such as foam or fabric. No hydrogel is necessary to couple the material to the skin. Mechanical contact can be provided by applying an elastomeric band around the MIEC material or using an elastomer or adhesive as the polymer in the MIEC material. This technology can address motion artifacts in recording electrodes that occurs due to squeeze out of the hydrogel.

The materials are flexible and conformal to the user and to other apparel and garments with which they may interact. They are easy to don and doff, inexpensive to fabricate, and not sticky compared to hydrogel. The material can be cast into any desired shape, including a sleeve, or can be cast to create custom fits. They have great mechanical and electronic compatibility with other apparel and garments as well.

The MIEC material produces a low impedance when contacting the skin to deliver a comfortable stimulus. This maximizes electrical stimulation with a lower current output. The materials are reusable, very durable, and return to their original shape post use. As a result, the use of hydrogel, conductive lotions, conductive gels, and rigid metal electrodes can be reduced or eliminated.

The materials, devices, and systems of the present disclosure will further be illustrated in the following non-limiting examples, it being understood that these examples are intended to be illustrative only and that the disclosure is not intended to be limited to the materials, conditions, process parameters and the like recited herein.

### EXAMPLES

### Example 1

Two different MIEC solutions were made.

The first MIEC solution was made from an HA solution containing 0.47 wt % of Sigma-Aldrich hyaluronic acid dispersed in water (w/w), an SWNT solution containing TUBALL single walled carbon nanotubes (0.2 wt. % SWNT's dispersed in water and carboxymethyl cellulose (w/w)), which acts as the electronic conductor, and a 37.5% solution of NBR Latex (Nipol LX330L), which acts as the matrix and insulative component of the MIEC material. These three solutions were mixed together to create a medium-viscosity liquid solution containing 49 wt. % of the HA solution, 23 wt. % of the SWNT solution, and 28 wt % of the NBR Latex solution. The ratio of hyaluronic acid to SWNTs was 5:1 (w/w). The Sigma-Aldrich hyaluronic acid had an Mw of 1,500,000 to 1,800,000 Da.

The second MIEC solution was made from an HA solution containing 0.71 wt. % of Medium Molecular Weight Making Cosmetic Grade hyaluronic acid dispersed in water (w/w), an SWNT solution containing TUBALL single walled carbon nanotubes (0.2 wt. % SWNT's dispersed in water and carboxymethyl cellulose (w/w)), and a 37.5% solution of NBR Latex (Nipol LX330L). These three solutions were mixed together to create a medium-viscosity liquid solution containing 49 wt. % of the hyaluronic acid solution, 23 wt. % of the SWNT solution, and 28 wt % of the NBR Latex solution. The ratio of hyaluronic acid to SWNTs was 7.5:1 (w/w). The Medium Molecular Weight Making Cosmetic Grade hyaluronic acid had an Mw of 100,000 to 500,000 Da.

Next, a composite material was made from each solution. A small layer of the MIEC solution was laid down the center of the mold. A textile material was placed on top of the solution so the solution wets out and the mold has a thin layer of solution wet out on it. The remaining MIEC solution was poured on top of the textile material. The textile material was stretched axially to help the solution to soak into the fabric. The textile material was then cured at room temperature for 72 hours or 38° C. for 24 hours.

The resulting composition, after curing, was found to induce good stimulation, transcutaneous electrical nerve stimulation (TENS) and evoking movement through neuromuscular electrical stimulation (NMES), FES, haptic, and EMG recording.

### Example 2

The procedure of Example 1 was followed to prepare the MIEC precursor mixture. A piece of low-density or high-density polyurethane (LDPU or HDPU) foam was then soaked in the solution and compressed and released around ˜100 times in the solution. Alternatively, the MIEC solution was poured into a plastic bag, the foam was placed in the bag, and the bag was moved around until the outer layer of the foam was homogenously black. The foam was squeezed or compressed ˜100 times slowly, so that there were not many bubbles present. The bag sat for 1 hour and the foam was then taken out. Both procedures allowed the MIEC solution to fill in every potential surface and cavity of the foam. This also allows for full internal and external coverage of the foam with the MIEC solution. The foam sample was cured for 5 days at room temperature or in an oven at 38° C. for 24-48 hours. The foam could be tested for complete curing by compressing—if no liquid evacuated, then it was fully cured. If liquid was present, additional curing time was needed.

### Example 3

The procedure of Example 1 was used to prepare two homogeneous MIEC solutions. Each solution was filtered through a medium mesh paint filter, then poured into a custom designed Delrin® mold. Any air bubbles present were removed, and the molds were cured for 72 hours at room temperature (20° C.-22° C.). Once fully cured, a corner of each MIEC sheet was lifted, and isopropyl alcohol was used to release the sheet from the mold. Borders along the edges were folded over to reinforce the edges. This provided a soft mixed ionic-electronic conductive (MIEC) material in the form of a long-lasting, reusable sheet.

Next, an electronic stimulation garment was provided which included a fabric layer and electrodes attached to the fabric layer. Pros-Aide® adhesive was applied to the fabric layer and permitted to dry. The reinforced edges of the MIEC sheet were then pressed against the dried adhesive

### Example 4

A MIEC sheet was used together with an electronic stimulation garment and tested with functional electrical stimulation (FES) to determine if it prevents “biting” after repeated use for 5 weeks. During testing with 3 able-bodied participants, biting did not occur when the MIEC sheet was used in combination with Signa Spray on the arm. The MIEC sheet+Signa Spray combination was found to prevent biting while evoking strong muscle contractions over the course of 5 weeks of testing. The data suggested that further testing may be required for use of the MIEC sheet with Signa Spray for periods greater than 5 weeks.

The soft MIEC sheet was able to induce movement with Signa Spray on the participants' arm around 3 mA, which is similar to the performance of a hydrogel sheet. The intensity of the stimulation/movement was greater than the state-of-the-art hydrogel sheet.

### Example 5

Three different MIEC sheets were made using different elastomers and tested for their mechanical properties. A Basic Tensile Test was performed with an extensometer on an Instron. The measuring parameters were as follows: Speed Rate=2.00 in/min, Data Rate (points per second)=20.00, and a 1-inch gauge length. The results are shown in Table 1 below:

TABLE 1

| | | | | | | | Max | |
|---|---|---|---|---|---|---|---|---|
| | Modulus | | Maximum | | UTS | | Breaking | |
| | (MPa) | | Strain (%) | | (MPa) | | Force (N) | |
| Elastomer | Avg | StDev | Avg | StDev | Avg | StDev | Avg | StDev |
| Nipol | 256.01 | 77.02 | 138.91 | 22.97 | 0.90 | 0.02 | 1.18 | 0.09 |
| LX55L | | | | | | | | |
| (control) | | | | | | | | |
| Nipol | 219.88 | 41.40 | 136.91 | 29.52 | 1.04 | 0.03 | 1.34 | 0.17 |
| LX370 | | | | | | | | |
| Nychem | 46.07 | 31.33 | 462.01 | 151.27 | 0.88 | 0.09 | 1.16 | 0.21 |
| 1570X79A | | | | | | | | |

The mechanical properties show that the MIEC material is soft (low Young's modulus), elastic (high strain), and robust. The MIEC material outperforms many hydrogels due to its flexibility, allowing more natural movement with great functionality. Electrical properties are similar to that of a hydrogel sheet, with low impedance and good electrical conductivity.

### Example 6

A mixed ionic-electronic conductor (MIEC) sheet was compared to other conduction enhancers and tested with functional electrical stimulation (FES) using an electronic stimulation garment (the NeuroLife® Sleeve and StimHub) to determine whether biting was prevented. During testing with 3 able-boded participants, biting did not occur when the MIEC sheet was used in combination with Signa Spray on the arm, even over an 8-hour test period and when the sleeve was simply draped over the arm without zipping up the zipper.

Seven conduction enhancer options for the sleeve were compared, specifically comparing impedance measurements with the sleeve both donned and doffed on the subjects' forearms and frequency of subject reported “biting” during FES while the sleeve was donned. The conduction enhancers tested were as follows:

1. No Conduction—Stainless steel electrodes directly in contact with skin;

2. Signa Spray—Signa spray applied to the arm (12 sprays then rubbed in with other hand);

3. AG735—Off-the-shelf hydrogel sheet (Axelgaard AG735) placed directly on the inside of the sleeve;

4. SRM—Off-the-shelf hydrogel sheet (Sekisui Plastics SR-M) placed directly on the inside of the sleeve;

5. GCR—Off-the-shelf hydrogel sheet (Sekisui Plastics G-CR 240-100-03) placed directly on the inside of the sleeve;

6. MIEC—MIEC sheet that is placed on the inside of the sleeve; and

7. MIEC+Signa Spray—MIEC sheet placed on the inside of the sleeve in combination with Signa Spray applied to the arm (12 sprays)

For sheet testing, one of the conduction enhancing sheets was attached to the inside of the sleeve. The sleeve was attached to the StimHub and impedance measurements were collected with the sleeve open face. Conductive spray was applied, if specified, to the arm (12 sprays). The sleeve was donned with the zipper aligned with the subject's ulna. Once donned, the arm was held in a neutral position to obtain impedance measurements and subjective reports of “biting” during FES.

For some tests, the sleeve was donned for 8 hours. Impedance measurements and subjective reports of “biting” during FES were recorded every hour. Measurements were taken with the participant's arm in a neutral position.

For loose fit testing, Signa Spray was applied to the arm (12 sprays) and the sleeve with MIEC sheet attached was rested on the subject's forearm and the sleeve was not zippered closed. This simulated an extremely loose fit with electrodes lifted or not contacting the arm.

For each of the conduction enhancer options detailed in the “Description of Test Setup” section, two measurements were conducted:

An impedance check of the electrode array in the sleeve was run for all the conduction enhancers options. Impedance was measured between a central electrode in the array and every individual electrode. Impedance checks were run at 1 mA and the resulting data was saved.

To evaluate biting, a program was run that simulates a cluster of anodes and a cluster of cathodes while each cluster is moved spatially around the array of electrodes, to provide a quick sweep of all electrodes. Notes were taken to report subjective biting, subjective strength of muscle contractions, and audible noises from the sleeve. The strength of muscle contractions was reported based on the sensations experienced by the participants as well as the observed movement if applicable. Electrode sweeps were run successively with increasing current amplitude until the muscle contractions became so strong that they were uncomfortable or biting was felt. The minimum stimulation current provided was 1 mA and the maximum was 8 mA. Note this is not the total current. The stimulation waveform was a rectangular wave with 500 psec Phase 1 and 1000 psec Phase 2 at 50 pulses/sec.

Both measurements were run on each conduction enhancer when the sleeve was both open faced, and on a participant's arm in a neutral position (except MIEC+Signa Spray which could only be run on the arm since the spray is applied directly to the arm). Open faced impedance checks measure impedances from the electrodes to the sheet and longitudinally through the sheet. Impedance checks on the arm include the impedance longitudinally through the sheet in parallel with the contact impedance of the sheet to skin and then through the tissue. The collected impedance data was processed in MATLAB.

Conclusion: From the comparison testing, the “MIEC+Signa Spray” conduction enhancer option was found to perform similarly to the “AG735” hydrogel sheet in that it prevented biting while evoking strong muscle contractions, but it was found to be much more usable than the “AG735” hydrogel sheet. Additionally, the “MIEC+Signa Spray” conduction enhancer option prevented biting during both types of testing. The average impedance measurements for the “MIEC+Signa Spray” was comparable to that of the “AG735” hydrogel sheets, though higher, and maintained the impedance with minimal change over an 8-hour period. Given that the “MIEC+Signa Spray” reliably prevented biting in all test cases and appeared stable over an 8-hour wear period, it is unlikely that biting will occur during its use, even in scenarios of lifting electrodes, bunching, or loose fit.

### Example 7

The MIEC sheet (no substrate), after molding, had a shiny side and a dull side. Some testing was performed to determine whether the orientation of the MIEC sheet relative to the electronic stimulation garment had any effect. When the shiny side of the MIEC sheet was on the electrodes, greater comfort was reported compared to the shiny side being on the skin. Larger muscle contractions and lower impedance were also measured.

Composite materials with the MIEC material impregnated into a textile substrate in two different ways were also tested. After curing, the composite material also had a shiny side and a dull side.

For the first composite material, the substrate was coated on both sides. The impedance data was better when the shiny side was on the skin, but greater comfort was reported when the shiny side was on the electrodes.

For the second composite material, the substrate was coated on one side and painted on the other side. For this embodiment, greater comfort, larger muscle contractions, and lower impedance occurred when the shiny side of the MIEC sheet was on the electrodes.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A composite material, comprising:
an electrically conductive material formed from polymer particles, an electrical conductor, and an ionic conductor; and
a textile or foam substrate.

2. The composite material of claim 1, wherein the electrically conductive material forms an electrically conductive layer that is directly adhered to the textile or foam substrate.

3. The composite material of claim 2, wherein the electrically conductive layer comprises a lip projecting from a surface of the electrically conductive layer or comprises a border around a perimeter of a surface of the electrically conductive layer.

4. The composite material of claim 1, wherein the textile or foam substrate is impregnated with the electrically conductive material.

5. The composite material of claim 1, wherein the polymer particles comprise acrylonitrile butadiene rubber (NBR), natural rubber, silicone, silicone acrylic, polyurethane, polyvinylidene fluoride, polyvinylidene chloride, or a fluoropolymer.

6. The composite material of claim 1, wherein the electrical conductor comprises carbon nanotubes, graphene or graphite structures, metal nanowires, and combinations thereof.

7. The composite material of claim 1, wherein the ionic conductor comprises glycosaminoglycans, fluorosulfonic acids, sulfated polysaccharides, phosphonic polyvinylsulfonic acids, graphene oxide, modified graphene oxide, and combinations thereof.

8. The composite material of claim 1, wherein a mass ratio of the ionic conductor to the electrical conductor is from 0.5 to 10.

9. The composite material of claim 1, wherein the ionic conductor comprises a glycosaminoglycan, and the electrical conductor comprises carbon nanotubes.

10. The composite material of claim 1, wherein the electrically conductive material further comprises an adhesive or a dispersant.

11. The composite material of claim 1, wherein the textile or foam substrate comprises cheesecloth, cotton, a polymer, a woven fabric, or a non-woven fabric; or wherein the textile or foam substrate is an open-cell foam.

12. The composite material of claim 1, wherein the electrical conductors are aligned to obtain anisotropic conductivity.

13. The composite material of claim 1, wherein the composite material is in the form of a sleeve, or wherein the composite material is in the form of a sheet having a length of at least 10 cm and a width of at least 10 cm.

14. A garment comprising:
a first textile layer having a plurality of electrodes attached thereto; and
the composite material of claim 1 attached to the first textile layer;
wherein the composite material covers the electrodes in the plurality of electrodes.

15. The garment of claim 14, wherein the composite material is attached to the first textile layer by an adhesive, or by sewing, or by a hook-and-loop fastener.

16. The garment of claim 14, having a form factor of a sleeve, vest, or a skullcap.

17. A method for making a composite material, comprising:
preparing a solution comprising an elastomeric phase, an electrical conductor, and an ionic conductor;
soaking a textile or foam substrate in the solution; and
curing the soaked textile or foam substrate to obtain the composite material.

18. The method of claim 17, further comprising compressing and releasing the textile or foam substrate during soaking.

19. An electrically conductive material formed from polymer particles, an electrical conductor, and an ionic conductor.

20. A garment comprising:
a first textile layer having a plurality of electrodes attached thereto; and
the electrically conductive material of claim 19 attached to the first textile layer;
wherein the electrically conductive material covers the electrodes in the plurality of electrodes.
